(19) United States
(12) Patent Application Publication
(10) Pub. No.: US 2002/0012634 A1
(43) Pub. Date: Jan. 31, 2002

(54) Antimicrobial dental materials contraining 2,4,4' -trichloro2' -hydroxydiphenyl ether

(75) Inventors: Kai Pflug, Konstanz (DE); Michael J. Noack, Koeln (DE)

Correspondence Address: Douglas J. Hura, Esquire, DENTSPLY INTERNATIONAL INC., 570 West College Avenue, York, PA, 17405, US

(73) Assignee: DENTSPLY DeTrey GmbH

(21) Appl. No.: 09/770,005

(22) Filed: Jan. 25, 2001

Related U.S. Application Data
(63) Continuation of application No. 09/543,266, filed on Apr. 5, 2000, now abandoned.
(60) Provisional application No. 60/044,995, filed on Apr. 28, 1997.

(51) Int. Cl. A61K 7/16

(52) U.S. Cl. 424/49

(57) ABSTRACT

Polymerizable denial materials having an antimicrobial effect are provided. These include dental materials such as protective dental varnishes, composites, compomers, fissure sealants, dental cements, dental bonding agents and similar materials, and containing 2,4,4′-trichloro-2′-hydroxydiphenyl ether.

### RELATED APPLICATION

This application claims the benefit of U.S. Provisional application Ser. No. 60/044,995 filed on Apr. 28, 1997.

### TECHNICAL FIELD

The invention relates to polymerizable dental materials. More particularly, the invention relates to such materials having an antimicrobial effect. Specifically, she invention relates to dental materials such as protective dental varnishes, composites, compomers, fissure sealants, dental cements, dental bonding agents and similar materials, and containing 2,4,4′-trichloro-2′-hydroxydiphenyl ether.

### BACKGROUND OF THE INVENTION

The relationship between bacterial flora and the development of periodontal disease and caries has been proven in a large number of publications (P. Axelsson et. al. in: J. Clin. Perio. 5, 133-151 (1978)]. To achieve reduction of these dental diseases it is therefore necessary to control the bacterial flora.

The most widely used approach to date to control the bacterial flora in the oral cavity has been mechanical cleaning methods such as brushing the teeth. Although this method has proved to be fairly successful in treating individuals, there is still a high recurrence raze. There is also Me problem of motivating people to good oral hygiene habits that they will maintain throughout their lives.

A variety of materials have been developed to suppress oral microorganisms. These include mouthrinses, dentifrices and gels containing antibacterial agents such as chlorhexidine and quarternary ammonium salts. These materials only offer a short-term antimicrobial effect.

Sustained release of an antimicrobial agent has been achieved by embedding chlorhexidine in a polymer to form a varnish. However, the materials developed so far display some disadvantages. For example, reported side effects of chlorhexidine, including staining and altered taste perception have limited its acceptance as attempts to reduce these side effects by using dilute solutions and flavoring agents have only been partly successful.

More importantly, these chlorhexidine varnishes are only effective for a limited period of time as the uncrosslinked polymer matrix does not prevent the antimicrobial agent from leaching out within a few days. For example, U.S. Pat. No. 4,496,322 discloses a dental varnish which contains chlorhexidine acetate, a benzoin gum, and an orally acceptable solvent. The composition, once applied to the teeth, is allowed to dry thereon and gives a film which provides sustained release of the antimicrobial agent over a period of a few days.

PCT WO 89/10736 describes dental glasspolyalkenoate cements made soluble in oral fluids by the addition of chlorhexidine. However, these materials dissolve after 1-4 weeks on the teeth and therefore are not suitable as long-term dental materials.

The broad spectrum antimicrobial agent 2,4,4′-trichloro-2′-hydroxydiphenyl ether, also known as “triclosan” has been known for more than 25 years. It has been used extensively in soaps, hand disinfectants, deodorants, laundry products, textile treatment, detergents, foot powders, shampoos and disposable paper products. It is soluble in many organic solvents, stable to hydrolysis and regarded as safe for humans and the environment. Triclosan is a highly effective antimicrobial with a broad spectrum of activity against both Gram-positive and Gram-negative bacteria as well as fungi, yeasts and viruses [Ciba-Geigy: Irgasan, Important toxicological and ecological data, 2512 E; Ciba-Geigy: Irgasan MP: General information. on chemical, physical, microbiological and toxicological properties]. In long-term experiments, no development of bacterial resistance to triclosan was found [C. L. Jones et. al., in: J. Dent. Res. 67, 46-50 (1988)].

More recently triclosan has also started to be used in oral care products, e.g. toothpastes and mouthrinses. Colgate Palmolive Company have employed triclosan as a toothpaste ingredient that has been proven to be effective against plaque bacteria [Bolden T. E. et al. in: J. Clin. Dent. 4, 125-131 (1992)]. Dentifrices containing triclosan have been tested and found to reduce plaque [K. W. Stephen et. al., in: J: Periodontal. 61, 674-679 (1990)].

### OBJECTS OF THE INVENTION

It is the object of the invention to provide polymerizable dental materials.

It is an additional object of the invention to provide such materials which promote a reduction in caries and other dental diseases related to microorganisms.

It is a further object of the invention to provide such materials having an antimicrobial agent, thereby providing an antimicrobial effect.

It is another object of the invention to provide such dental materials having physical properties similar to those materials without the antimicrobial agent.

These and other objects so the invention which will become apparent from the discussion to herein, are accomplished by the invention as hereinafter described and claimed.

### BRIEF SUMMARY OF THE INVENTION

The above-mentioned objects can be accomplished by adding from about 0.001 to about 20 percent by weight of the broad spectrum antimicrobial agent triclosan to otherwise dental materials. As the water solubility of triclosan is low and it is embedded in a crosslinked polymer matrix, leaching of the triclosan is low, resulting in a long-term antimicrobial effect. Incorporation of efficacious amounts of triclosan does not affect the mechanical properties of the dental materials.

### PREFERRED EMBODIMENTS FOR CARRYING OUT THE INVENTION

The present invention describes polymerizable dental materials that have an antimicrobial effect due to the incorporation of 2,4,4′-trichloro-2′-hydroxydiphenyl ether into the composition. Curable dental materials with an antimicrobial effect are provided for prophylactic and restorative treatment of teeth, including those materials intended for use with enamel, dentin, dental metals and the like.

The dental materials according to the invention preferably contain a matrix of curable or hardenable resin material or materials. Such materials include for example, methacrylate compounds (preferably dimethacrylate), urethane compounds and the like. Any conventional dental resin or curable dental matrix material is within the scope of the invention. The dental materials may also contain fillers, fluoride, stabilizers, initiators, solvents and other substances conventionally used in dental materials.

As will be demonstrated hereinbelow, the incorporation of triclosan into dental materials causes them to have antimicrobial properties. These antimicrobial properties lead to a reduction in caries and other dental diseases related to microorganisms. As dental materials are usually employed in situations where the tooth is either endangered or already damaged, the incorporation of triclosan into these materials has the additional advantage of getting the antimicrobial exactly to the location in the oral cavity where it is most needed or desired.

In the curable dental materials described in this invention, the antimicrobial agent triclosan is embedded in a polymeric matrix. This provides the dental materials with a long-lasting antimicrobial effect as the triclosan cannot leach out of these materials quickly. This aspect of the invention will be demonstrated hereinbelow.

The incorporation of triclosan can be employed in dental bonding agents, composite restorations, compomer restorations, fissure sealants or other conventional dental materials for which an antimicrobial effect is desirable. It has been unexpectedly found that incorporation of sufficient amounts of triclosan into these dental materials does not detrimentally affect the mechanical properties of the materials.

Polymerizable dental materials, as briefly discussed above, are materials that form a polymer upon hardening. The mechanism of the polymer formation may be initiated chemically or by irradiation (e.g. with visible light). The chemical curing may occur by radical polymerization or by an acid-base-reaction. Polymerizable dental materials comprise composites, compomers, fissure sealants, dental cements, dental bonding agents and similar materials.

These dental materials are made to have an antimicrobial effect by incorporation of from about 0.001 to about 20 percent by weight of triclosan. The triclosan is preferably added in the unpolymerized state of the dental materials. After curing, a polymeric network is formed that does not only harden the dental material but also serves as a matrix for the triclosan, embedding it in a way that prevents rapid leaching. This polymeric network ensures the long-term antimicrobial efficacy of the triclosan.

General Experimental

The following examples are given to further illustrate the present invention. To demonstrate the invention, a dental protective varnish, a composite dental restorative material and a dental bonding agent were prepared, each containing various amounts of triclosan. It is understood, however, that the invention is not limited by these examples, and that other dental materials are also within the scope of the invention as was discussed hereinabove. Each of the illustrative inventive examples below was tested for its antimicrobial effect, leaching propensity and/or for their relevant physical or mechanical properties.

### EXAMPLE 1

### Antimicrobial Protective Varnish

An antimicrobial protective varnish for exposed dentin was prepared containing the following components.

### Example 1 Composition

80 wt % ethanol

10.5 wt % UDMA-resin (2,7,7,9,15-pentamethyl-4,13-dioxo-3,14-dioxa-5,12-diaza-hexadecan-1,16-diyldimethacrylate)

4.8 wt % PENTA (dipentaerythritol pentaacrylate monophosphate)

3.0 wt % urethane resin R5-62-1 (7,7,9,63,63,65-Hexamethyl-4,13,60,69-tetraoxo-3,14,19,24,29,34,39,44,49,54,59,70-dodecanoxa-5,12,61,68-tetraaza-doheptaconta-1,72-diyldimethacrylate)

0.6 wt % ethyl 4-dimethylaminobenzoate

0.1 wt % 2,6-di-tert-butyl-p-cresol

0.2 wt % cetylamine hydrofluoride

0.6 wt % trimethylolpropane trimethacrylate

0.2 wt % camphorquinone.

To this mixture (100 wt %), various amounts of triclosan as mentioned below were added. This varnish had a low viscosity and deeply penetrated the dentin. After application, the solvent was removed by air-drying. Curing was done with a dental curing lamp with visible light for 20 seconds. A thin, strong polymeric film (thickness approximately 2-6 microns) remained.

Antimicrobial tests

In in-vitro rests, a film of the composition above (2 wt % triclosan) was shown to have an antimicrobial effect on streptococcus mutans as follows:

Test plates were filled with approximately 50 μl of an antimicrobial varnish composition according to Example 1. As a reference, similar formulations were prepared not containing fluoride and/or triclosan but with an otherwise unchanged composition. The solvent ethanol was evaporated under nitrogen and the varnish was light cured under nitrogen to prevent incomplete polymerization due to oxygen inhibition.

These test plates were filled with 50 μl of a liquid containing approximately 5×10⁴ CFU of streptococcus mutans in PBS+10% serum. Contact time was 30 seconds, 10 minutes (min), 1 hour (h), 3 hours and 6 hours at 37° C. An unfilled test plate was used as negative control. Each test was run three times. Subsequently the test solution was transferred to a new plate and subjected to enrichment. An MTT test was carried out to detect living streptococci mutans.

This test was run on two different days. Tables I and II show the results obtained.

TABLE I

| test | fluoride | triclosan | growth | inhibition | (%) after | | |
|---|---|---|---|---|---|---|---|
| plate | (wt %) | (wt %) | 30 sec | 10 min | 1 h | 3 h | 6 h |
| 1 | 0 | 0 | 3 | 3 | 0 | 17 | 20 |
| 2 | 0.2 | 0 | 2 | 0 | 2 | 13 | 31 |
| 3 | 0 | 2 | 0 | 37 | 100 | 100 | 100 |
| 4 | 0.2 | 2 | 4 | 12 | 100 | 100 | 100 |

TABLE II

| test | fluoride | triclosan | growth | inhibition | (%) after | | |
|---|---|---|---|---|---|---|---|
| plate | (wt %) | (wt %) | 30 sec | 10 min | 1 h | 3 h | 6 h |
| 1 | 0 | 0 | 9 | 0 | 0 | 20 | 26 |
| 2 | 0.2 | 0 | 0 | 0 | 4 | 20 | 41 |
| 3 | 0 | 2 | 17 | 52 | 100 | 100 | 98 |
| 4 | 0.2 | 2 | 14 | 71 | 100 | 100 | 100 |

These tests show that the antimicrobial varnish formulations containing triclosan have a high efficacy with regard to effect on streptococcus mutans.

To show that an antimicrobial effect is still present after elution of the material, the test was repeated with the same test plates after pre-elution in 0.9% NaCl for 7 days at 37° C. Though the antimicrobial efficacy is somewhat lower, it still is significant in the triclosan-containing test plates. Results are reported in Table III.

TABLE III

| test | fluoride | triclosan | growth | inhibition | (%) after | | |
|---|---|---|---|---|---|---|---|
| plate | (wt %) | (wt %) | 30 sec | 10 min | 1 h | 3 h | 6 h |
| 1 | 0 | 0 | 0 | 0 | 0 | 0 | 0 |
| 2 | 0.2 | 0 | 0 | 0 | 3 | 0 | 0 |
| 3 | 0 | 2 | 0 | 34 | 53 | 67 | 72 |
| 4 | 0.2 | 2 | 9 | 1 | 21 | 39 | 63 |

Leaching tests

To demonstrate the low leaching raze of triclosan despite its antimicrobial efficacy in the varnish, plaques of approximately 1.2 g (width 2 mm, diameter 25 mm) were made from a mixture of the varnish components of Example 1, except for the solvent ethanol (triclosan content 6.25 wt % based on resin mixture as described above). These plaques were light-cured and stored in artificial saliva (Ringer solution) for 20 days at 37° C. By UV/Vis spectroscopy, no triclosan could be found in the artificial saliva. Control experiments demonstrate that this indicates that less than 0.1% of the total amount of triclosan embedded in the plaque had leached out. However, fluoride contained in the plaques does leach out, probably due to the smaller size of the fluoride ions.

The low triclosan leaching was also proven by a different experiment. Plaques as described above were thermocycled 500 times (5° C. and 55° C., 20 seconds each). Weighing before and after thermocycling showed a weight difference of +1% (absorption of some water) and not the loss of 6.25% to be expected if all the triclosan had leached out.

The experiments measuring the triclosan leaching of plaques were repeated with a mixture containing a significantly higher triclosan content (40 wt % based on resin mixture). Again, plaques were made from a mixture of the varnish components except for the solvent ethanol.

These plaques were light-cured and stored in artificial saliva (Ringer solution) for 14 days at 37° C. By UV/Vis spectroscopy, some triclosan could be found in the artificial saliva. Calibration showed that this corresponded to a leaching of only 0.2% of the overall triclosan content of the plaque.

Mechanical properties

To demonstrate the effect of triclosan on the hardness of the varnish, plaques of approximately 1.2 g (width 2 mm, diameter 25 mm) with varying triclosan contents (wt % based on resin mixture) were made from a mixture of the varnish components of Example 1, except for the solvent ethanol. Different mixture ratios of the resins were used. The plaques were light-cured, and Barcol hardness was measured.

The hardness of the antimicrobial varnish containing low triclosan concentrations was found to be as high as the hardness of the varnish not containing any triclosan. Only at higher triclosan concentrations the hardness of the varnish dropped.

Thermocycling (500 cycles, 20 seconds at 5° C., 20 seconds at 55° C.) does lower hardness somewhat, but not significantly more than with the formulation not containing any triclosan. Mechanical test results are reported in Table IV.

TABLE IV
hardness of cured resin formulations (Barcol
hardness 934-1)

| | | | Hardness before/after |
|---|---|---|---|
| Code | Triclosan | Resin base | thermocycling (wt %) |
| 1 | — | | 50/46 |
| 2 | 10 | KP2-15-2 | 43/38 |
| 3 | 15 | KP2-15-2 | 33/32 |
| 4 | 20 | KP2-15-2 | 36/31 |
| 5 | 25 | KP2-15-2 | 27/23 |
| 6 | 30 | KP2-15-2 | 12/<10* |
| 7 | 40 | KP2-15-2 | <10/<10 |
| 8 | — | | 40.7 ± 0.7 |
| 9 | 4 | KP2-55 | 41.5 ± 0.7 |
| 10 | 6 | KP2-55 | 39.5 ± 1.6 |
| 11 | 8 | KP2-55 | 39.0 ± 1.5 |
| 12 | 10 | KP2-55 | 35.7 ± 0.8 |
| 13 | 15 | KP2-55 | 35.4 ± 1.7 |

* “<” means “less than”

### EXAMPLE 2

Antimicrobial Composite

A composite restorative material was mixed from 73.7% glass filler (Barium Aluminum Corning 7724 glass silanated with γ-methacryloyloxypropyltrimethoxysilane) and 26.3% resin matrix. The resin matrix was composed of the following materials.

### Example 2 Composition

98.582 wt % EBPADMA urethane resin (ethoxylated bisphenol-A-dimethacrylate urethane resin)

0.025 wt % 2,6-di-tert-butyl-p-cresol

0.163 wt % camphorquinone

0.4 wt % 2-hydroxy-4-methoxybenzophenone

0.65 wt % N-methyl-diethanolamine

0.018 wt % 2.5-dihydroxyterephtalic acid diethylester

0.081 wt % triethyleneglycol dimethacrylate

0.081 wt % bisphenol-A-dimethacrylate

Various amounts of triclosan were incorporated by dissolving the triclosan in the resin matrix before mixing filler and resin.

Antimicrobial effect

In in-vitro tests, the hand-mixed composite restorative material containing various amounts of triclosan were shown to have an antimicrobial effect on streptococcus mutans.

Test plates were each filled with a single cylindric sample (diameter 5 mm, height approximately 2 mm) of cured composite material. These test plates were filled with 50 μl of a liquid containing approximately 5×10⁴ CFU of streptococcus mutans in PBS+10% serum. Contact time was 30 seconds, 10 min, 1 hour, 3 hours and 6 hours a 37° C. An unfilled test plate was used as negative control. Each test was run three times. Subsequently the test solution was transferred to a new plate and subjected to enrichment. An MTT test was carried out to detect living streptococci mutans.

The test was repeated with the same samples after sterilization and 7 d pre-elution with 0.9% aqueous NaCl— solution at 37° C. (see Table V, second elution). The materials showed a marked antimicrobial effect that rises with triclosan content and that even increased after pre-elution.

TABLE V
Antimicrobial properties of experimental dental
composite

| | | | Growth inhibition in % after | | | |
|---|---|---|---|---|---|---|
| | Triclosan | | contact time | | | |
| Code | (wt % *) | Elution | 10 min | 1h | 3h | 6h |
| 1 | 5 | first | 0 | 3 | 7 | 17 |
| 2 | 10 | first | 0 | 23 | 34 | 41 |
| 3 | 10 | second | 0 | 41 | 95 | 100 |
| 4 | 15 | first | 16 | 30 | 100 | 100 |
| 5 | 15 | second | 61 | 99 | 100 | 100 |

* based on matrix

Mechanical properties

The compressive strength of hand-mixed composite restorative materials as described above containing various amounts of triclosan was measured. Results are in Table V.

TABLE VI
Compressive strength of composite restorative
materials

| | Triclosan | Matrix | Glass | Comp. Strength |
|---|---|---|---|---|
| Code | (wt %*) | (wt %) | (wt %) | (MPa) |
| 1 | 0 | 26.3 | 73.7 | 278 ± 15 |
| 2 | 5 | 26.3 | 73.7 | 280 ± 15 |
| 3 | 10 | 26.3 | 73.7 | 276 ± 10 |
| 4 | 15 | 26.3 | 73.7 | 255 ± 9 |

* based on resin matrix

With this inventive composite restorative material, no change of compressive strength could be found up to 10% triclosan content in the matrix. 15% triclosan, however, led to some decrease in compressive strength.

### EXAMPLE 3

Antimicrobial Dental Bonding Agent

An antimicrobial dental bonding agent formulation containing triclosan was tested for adhesion and antimicrobial properties. The bonding agent was composed of the following materials.

### Example 3 Composition

80 wt % ethanol

10.5 wt % UDMA-resin (2,7,7,9,17-pentamethyl-4,13-dioxo-3,14-dioxa-5,12-diaza-hexadecan-1,16-diyldimethacrylate)

4.8 wt % 2PENTA (dipentaerythritol pentaacrylate monophosphate)

3.0 wt % urethane resin R5-62-1 (7,7,9,63,63,65-Hexamethyl-4,13,60,69-tetraoxo-3,14,19,24,29,34,39,44,49,54,59,70-dodecanoxa-5,12,61,68-tetraaza-doheptaconta-1,72-diyldimethacrylate)

0.6 wt % ethyl 4-dimethylaminobenzoate

0.1 wt % 2,6-di-tert-butyl-p-cresol

0.2 wt % cetylamine hydrofluoride

0.6 wt % trimethylolpropane trimethacrylate

0.2 wt % camphorquinone.

To this mixture (100 wt %), various amounts of triclosan as mentioned below were added.

Antimicrobial tests

In in-vitro tests, a dental bonding agent containing various amounts of triclosan was shown to have an antimicrobial effect on streptococcus mutans:

Test plates were filled with approximately 50 μl of the dental bonding agent composition comprising the substances above. The solvent ethanol was evaporated under nitrogen and the varnish was light cured under nitrogen to prevent incomplete polymerization due to oxygen inhibition.

These test plates were filled with 50 μl of a liquid containing approximately 5×10⁴ CFU of streptococcus mutans in PBS+10% serum. Contact time was 30 seconds, 10 min, 1 hour, 3 hours and 6 hours at 37° C. An unfilled test plate was used as negative control. Each test was run three times. Subsequently the test solution was transferred to a new plate and subjected to enrichment. An MTT test was carried out to detect living streptococci mutans.

To show that an antimicrobial effect is still present after elution of the material, the test was repeated with the same test plates after sterilization and pre-elution in 0.9% NaCl for 7 days at 37° C. (see Table VII, second elution). Though the antimicrobial efficacy is somewhat lower, it still is significant in the triclosan-containing test plates.

TABLE VII
antimicrobial effect of triclosan-
containing dental bonding agent
at first and second elution

| | Triclosan | | Growth inhibition (%) after contact time | | | |
|---|---|---|---|---|---|---|
| Code | (wt %) | Elution | 10 min | 1h | 3h | 6h |
| 1 | 2 | first | 0 | 0 | 7 | 36 |
| | | second | 5 | 6 | 13 | 30 |
| 2 | 4 | first | 0 | 0 | 20 | 42 |
| | | second | 5 | 22 | 27 | 35 |
| 3 | 6 | first | 15 | 25 | 31 | 50 |
| | | second | 14 | 35 | 33 | 43 |
| 4 | 8 | first | 20 | 45 | 57 | 71 |
| | | second | 18 | 30 | 39 | 45 |
| 5 | 10 | first | 52 | 64 | 79 | 99 |
| | | second | 29 | 59 | 67 | 98 |
| 6 | 15 | first | 75 | 100 | 100 | 100 |
| | | second | 52 | 76 | 100 | 100 |

* based on resin matrix

These results demonstrate that the antimicrobial dental bonding agents according to the present invention display a marked antimicrobial effect that is rising with rising triclosan content. Also, after elution the antimicrobial dental bonding agent still shows antimicrobial efficacy hat is only slightly lower than initially.

Mechanical properties

For the test of mechanical properties, a formulation using acetone as solvent (80 wt %) was used instead of ethanol. Otherwise the composition remained unchanged. Pretreatment before application of the antimicrobial bonding agent was with a conditioning solution (36% phosphoric acid gel). TPH Spectrum (Dentsply) was used as light-cure type composite resin.

Bond strength was determined by the shear bond strength of the composite resin in relation to enamel and dentin. Human molars were used. For purposes of enamel bond tests, the enamel surface of 6 human molars was polished with carborund (SiC). This fresh, dry enamel surface was treated with the etching solution for 20 seconds, followed by compressed air drying. Thereafter, the bonding agent was applied and, 20 seconds later, compressed air drying was effected. This coat was light-cured for 20 seconds, using a Spectrum curing light (Dentsply International Inc.). Subsequently, a plastic mold with an inner diameter of 5 mm and a height of 2 mm was fixed to the surface and TPH Spectrum was filled into the interior of the mold. The surface was subjected to visible light irradiation by the Spectrum curing light via the mold for 40 seconds. After light-curing, the teeth were stored at 37° C. for 24 hours, then thermocycled 500 times (20 seconds at 5° C., 20 seconds at 55° C.), embedded in gypsum and tested with a Zwick Z010/TN2A tabletop universal testing machine at a speed of 1 millimeter per minute (mm/min).

For purposes of dentin bond tests, the dentin surface of 6 human molars was exposed with a diamond saw and ground with #500 sandpaper. This fresh dentin surface was treated with the conditioner for 20 seconds, followed by careful drying with a paper towel. This drying should leave a dry-looking surface but should not be too harsh. Thereafter, the bonding agent was applied and, 20 seconds later, compressed air drying was effected. This coat was light-cured for 20 seconds, using a Spectrum curing light (Dentsply). Subsequently, a plastic mold with an inner diameter of 5 mm and a height of 2 mm was fixed to the surface and TPH Spectrum was filled into the interior of the mold. The surface was subjected to visible light irradiation by the Spectrum curing light via the mold for 40 seconds. After light-curing, the teeth were stored at 37° C. for 24 hours, then thermocycled 500 times (20 seconds at 5° C., 20 seconds at 55° C.), embedded in gypsum and tested with a Zwick Z010/TN2A tabletop universal testing machine at a speed of 1 mm/min.

TABLE VIII
Adhesion of an antimicrobial dental
bonding agent to dentin and enamel

| | content triclosan | Adhesion (MPa) to | |
|---|---|---|---|
| Code | (wt %) | Dentin | Enamel |
| 1 | — | 21.6 (15) | 15.4 (12) |
| 2 | 1 | 18.9 (20) | 16.6 (14) |
| 3 | 2 | 18.7 (17) | 16.8 (20) |
| 4 | 3 | 15.6 (59) | 16.9 (18) |

These tests show that inclusion of us to 3 wt % of triclosan into the dental bonding agent does not change adhesion to enamel. The adhesion values to dentin for the dental bonding agent containing 1% and 2% of triclosan are not significantly lower than those of dental bonding agent not containing triclosan. Only at higher triclosan concentrations the adhesion value drops significantly.

It is apparent therefore, that the antimicrobial dental compositions as described herein are effective in carrying out the objects of the invention. While the principles of the invention have been made clear by the illustrative embodiments discussed, those skilled in the art will appreciate that modifications to composition components, amounts, grades, process and method conditions and the like, can be made and still fall within the scope of the those principles. Specifically for example, dental materials other than those described and exemplified above can be rendered antimicrobial with desired leaching and mechanical characteristics, all of which fall within the scope of the invention.

1. A dental material comprising the antimicrobial agent 2,4,4′-trichloro-2′-hydroxydiphenyl ether.

2. A polymerizable dental material comprising the antimicrobial agent 2,4,4′-trichloro-2′-hydroxydiphenyl ether.

3. A dental material as in claim 1, comprising from about 0.01 to about 50 percent by weight of the antimicrobial agent 2,4,4′-trichloro-2′-hydroxydiphenyl ether.

4. A dental material as in claim 1, comprising from about 0.1 to about 30 percent by weight of the antimicrobial agent 2,4,4′-trichloro-2′-hydroxydiphenyl ether.

5. A dental material as in claim 1, comprising from about 0.5 to about 25 percent by weight of the antimicrobial agent 2,4,4′-trichloro-2′-hydroxydiphenyl ether.

6. A dental material as in claim 1, comprising from about 1 to about 20 percent by weight of the antimicrobial agent 2,4,4′-trichloro-2′-hydroxydiphenyl ether.

7. A dental material comprising a dental composition selected from the group consisting of varnishes, composites, compomers, sealants, dental bonding agents, and cements, and comprising from about 1 to about 20 percent by weight of the antimicrobial agent 2,4,4′-trichloro-2′-hydroxydiphenyl ether.

8. A dental material as in claim 7, wherein said dental composition further comprises a cross-linkable polymer, such that said 2,4,4′-trichloro-2′-hydroxydiphenyl ether is embedded in a crosslinked polymer matrix after curing of the dental composition.

9. A dental material as in claim 8, wherein said 2,4,4′-trichloro-2′-hydroxydiphenyl ether is prevented from leaching in an aqueous environment by said embedding.

10. A dental material as in claim 9, having an antimicrobial effect based upon the antimicrobial agent.

11. A dental material as in claim 9, wherein said dental material has structural properties substantially similar to those of the material without the antimicrobial agent 2,4,4′-trichloro-2′-hydroxydiphenyl ether.
